(19) United States
(12) Patent Application Publication
(10) Pub. No.: US 2024/0041464 A1
(43) Pub. Date: Feb. 8, 2024

(54) DEVICES, SYSTEMS, AND METHODS FOR OCCLUDING A BODY PASSAGE

(71) Applicant: BOSTON SCIENTIFIC SCIMED, INC., MAPLE GROVE, MN (US)

(72) Inventors: Sean P. Fleury, Princeton, MA (US); John Thomas Favreau, Shrewsbury, MA (US); Bryan Bannon, Duxbury, MA (US)

(73) Assignee: BOSTON SCIENTIFIC SCIMED, INC., MAPLE GROVE, MN (US)

(21) Appl. No.: 18/228,786

(22) Filed: Aug. 1, 2023

Related U.S. Application Data
(60) Provisional application No. 63/394,425, filed on Aug. 2, 2022.

(51) Int. Cl. A61B 17/12

(52) U.S. Cl. CPC A61B 17/12136; A61B 17/12031

(57) ABSTRACT

An implantable device configured to withstand forces imparted thereto by anatomical structure at a deployment site. The implantable device includes an outer portion and inner portion. The outer portion is positioned between the inner portion and the walls of the anatomical deployment site. The outer portion may enclose the inner portion. The outer portion may be a compliant balloon compressible in response to radially-inwardly directed anatomical forces exerted thereon. The inner portion may include one or more portions configured to retain the implantable device at the deployment site and to resist migration of the implantable device. The inner portion may be movable with respect to the outer portion. The inner portion may include more than one portions, such portions being movable with respect to each other. A shaft may extend through the device, such as to facilitate inflation of the outer portion and/or positioning of the inner portion.

### CROSS-REFERENCE TO RELATED APPLICATIONS

This application claims the benefit of priority under 35 U.S.C. § 119 to U.S. Provisional Application No. 63/394,425, filed Aug. 2, 2023, the entire disclosure of which is hereby incorporated by reference herein for all purposes.

### FIELD

The present disclosure relates generally to the field of devices, systems, and methods for occluding a body lumen.

### BACKGROUND

It may be desirable for a body passage to be occluded or blocked or plugged for any of a variety of reasons. For instance, it may be desirable to prevent passage of materials through the passage and divert the materials to pass through another lumen or passage, such as via a bypass or anastomosis. One type of procedure (known as a gastric bypass procedure) involves creating an anastomosis between the stomach and a portion of the small intestines downstream of the pylorus, such as a portion of the jejunum. Such procedure is intended to have ingested food bypass part of the small intestine (and absorption therein). Thus, another aspect of such procedure involves occluding the pylorus so that food does not enter the duodenum, and, instead, passes through the bypass anastomosis and into the jejunum to reduce the metabolic disease being treated by such procedure.

Various occlusion devices, such as for occluding a pylorus, are known in the art. However, some occlusion devices positioned in dynamic environments such as the pylorus may show wear and/or even fatigue after prolonged use. For instance, pyloric occlusion devices are subjected to constant pressure as the stomach contracts in coordinated waves (gastric slow waves beginning in the fundus, propagating toward the antrum, and eventually slamming the pyloric sphincter shut) during digestion—approximately every twenty seconds. A pyloric occlusion device generally is positioned in the most muscular region of the stomach and must be able to resist regular muscular contractions applied thereto while also resisting migration. Improvements to occlusion devices, systems, and methods would thus be welcome in the industry.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an implantable device configured to be delivered to and deployed at a deployment site includes an inner scaffold portion shiftable between a delivery configuration and an expanded deployed configuration; and an outer portion formed separately from the inner portion and positioned over the inner portion such that, upon deployment, the outer portion is positioned between a body wall at the deployment site and the inner portion of the implantable device.

In some embodiments, the inner portion has a first inner portion and a second inner portion. In some embodiments, the first inner portion and the second inner portion are movable with respect to the outer portion and with respect to each other. In some embodiments the first inner portion and the second inner portion are movable away from each other in response to force applied to the outer portion.

In some embodiments, the outer portion is a balloon enclosing the inner portion. In some embodiments, an end of the balloon is tapered to guide the implantable device to a deployment site. In some embodiments, the balloon includes an inflation valve separably couplable with an inflation lumen to allow inflation of the balloon and separation from the inflation lumen upon deployment of the balloon.

In some embodiments, the inner portion has one or more ring-shaped retention members. In some embodiments, the inner portion has first and second toroidal retention members.

In some embodiments, the inner portion has one or more disk-shaped retention members.

In some embodiments, the implantable device further includes a shaft extending through the implantable device. In some embodiments, the shaft extends axially within the implantable device, and the inner portion has a first inner portion and a second inner portion movable with respect to the shaft in response to radially-inwardly directed force applied to the implantable device when the implantable device is deployed at a deployment site.

In some embodiments, the inner portion has one or more disk-shaped retention members having collars mounted on the shaft and movable with respect to the shaft.

In some embodiments, the outer portion is expandable upon shifting of the inner portion from the delivery configuration to the deployed configuration.

In accordance with various principles of the present disclosure, method of forming an implantable occlusion device includes mounting one or more retention members over a shaft; positioning a compliable cover portion over the retention members and the shaft; and sealing the cover portion with respect to the shaft at a proximal end and a distal end to enclose the retention members therein.

In some embodiments, the method further includes mounting the one or more retention members slidably over the shaft.

In accordance with various principles of the present disclosure, a method of occluding a body passage includes delivering an implantable occlusion device to the body passage, the implantable occlusion device having an inner scaffold portion and an outer portion enclosing the inner portion; expanding the outer portion to engage the wall of the body passage to deploy the implantable occlusion device and to occlude the body passage; allowing the inner portion to shift to an expanded deployed configuration on either side of the body passage to anchor the implantable occlusion device with respect to the body passage to resist migration from the deployment site.

In some embodiments, the method further includes deploying the implantable occlusion device across a pylorus.

In some embodiments, the outer portion is a compliant balloon and the inner portion includes first and second inner portions, and the method further including deploying the compliant balloon at a deployment site which applies radially-inwardly directed force to the compliant balloon to compress a section of the compliant balloon and to cause the first and second inner portions to move apart from each other axially.

In some embodiments, the outer portion is a balloon, and the method further includes inflating the balloon with an inflation lumen when the balloon is delivered to the deployment site, and removing the inflation lumen from the balloon to deploy the implantable occlusion device at the deployment site.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an implantable device formed in accordance with various aspects of the present disclosure and positioned in a schematic representation of a gastrointestinal environment.

FIG. 2A illustrates a perspective view of an example of an embodiment of an implantable occlusion device formed in accordance with various principles of the present disclosure.

FIG. 2B illustrates an elevational view of an example of an embodiment of an implantable occlusion device such as in FIG. 2A, with a force being applied to a medial section thereof.

FIGS. 3A-3C illustrate stages of an example of a manner of inflating a component of an implantable occlusion device formed in accordance with various principles of the present disclosure.

FIG. 4A illustrates an elevational view of another an example of an embodiment of an implantable occlusion device formed in accordance with various principles of the present disclosure.

FIG. 4B illustrates an elevational view of an example of an embodiment of an implantable occlusion device such as in FIG. 3A, in a schematic representation of an environment in which the implantable occlusion device may be implanted.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “channel” or “bore” is not limited to a circular cross-section. As used herein, a “free end” of an element is a terminal end at which such element does not extend beyond.

The present disclosure relates to devices, systems, and methods which may be positioned at an anatomical site (referenced herein as a deployment site for the sake of convenience and without intent to limit). Devices, systems, and methods of the present disclosure may be used to occlude a body passage or lumen. One example of such use is in connection with occluding a pylorus (e.g., in connection with creation of a gastrojejunal anastomosis). However, it will be appreciated that the present disclosure is not limited to such use (e.g., occlusion) and may have broader uses and/or configurations. A device used to occlude a body passage may be known by various terms which may be used interchangeably herein, such as an exclusion or obstruction device. Reference is made herein to an implantable occlusion device, or simply an implantable device, for the sake of convenience and without intent to limit. Deployment of the implantable occlusion device may be at or across or along an anatomical structure. It will be appreciated that terms such as at, across, along, etc., may be used interchangeably herein without intent to limit. The anatomical structure may be a body passage, lumen, etc., the present disclosure not being limited to use with a particular anatomical structure. For the sake of convenience, and without intent to limit, reference may be made herein simply to a deployment site.

In accordance with various principles of the present disclosure, an implantable occlusion device is formed with an outer portion and an inner portion. The outer portion may be in the form of a cover portion over the inner portion. In some embodiments, the cover portion is formed of a flexible material, such as a compliant material. In some embodiments, the cover portion is an enclosed structure such as a balloon. The inner portion may be structured to hold the implantable occlusion device in place with respect to an anatomical structure. For instance, the inner portion may be a scaffold type structure having a defined configuration/structure, in contrast with an element which may have a configuration determined by another element. More particularly, the inner portion may have a self-defined structure in contrast with a flexible compliant element such as an outer portion with a structure or configuration defined by an underlying structure as the inner portion. Such inner portion may be a scaffold, stent, frame, or the like, such terms being used interchangeably herein without intent to limit.

Whereas prior stents may have coatings, such coatings are formed on the stent (e.g., to protect body tissue from the stent, to prevent tissue ingrowth, etc.) and generally are not separate or separable from the stent. In contrast, the outer portion of an implantable occlusion device formed in accordance with various principles of the present disclosure may be a separate cover portion positioned over a stent, to be positioned between the anatomical structure along which the implantable occlusion device is to be deployed (e.g., a body passage) and the stent. As such, the inner portion may move with respect to the outer portion, which may provide an implantable occlusion device formed in accordance with various principles of the present disclosure with a greater degree of flexibility with regard to positioning and deployment than achieved by prior art devices. Moreover, a separately formed outer portion may have more structural impact than may achieved by prior art stents, such as by providing more of a barrier (e.g., a protective barrier) for the stent than provided by coatings or other features of prior art stents.

In some embodiments, one or both of the inner and outer portions of the implantable occlusion device are shiftable from a delivery configuration to a deployed configuration. In the delivery configuration, the implantable occlusion device may be in a compact configuration, such as compressed, constrained, restrained, etc. (such terms, in various grammatical forms thereof, being used interchangeably herein without intent to limit). A compact configuration generally allows delivery of the implantable occlusion device transluminally through the body (i.e., through body passages and without the need for open surgery). In the deployed configuration, the implantable occlusion device is expanded, such as to engage the wall of the body passage. For instance, one or both of the inner and outer portions of the implantable occlusion device are expandable. The outer portion may be expanded by expansion of the inner portion (such as in an embodiment in which the inner portion is self-expanding). Alternatively, if the outer portion envelopes and encloses the inner portion, an inflation lumen may be coupled with the outer portion to expand the outer portion. The inflation lumen may be removably coupled to allow deployment of the implantable occlusion device without the inflation lumen coupled thereto.

In some embodiments, the inner portion includes one or more retention members configured to anchor or otherwise hold the implantable occlusion device with respect to the deployment site. The retention members may be structured to have sufficient strength to retain the inner portion in place with respect to the anatomical walls of the deployment site and to resist a pull-out force on the device (such as induced by anatomical movements or processes, such as peristalsis if deployed in the gastrointestinal tract). The inner portion may be flexible, such as to conform to the shape of the deployment site at which the implantable occlusion device is deployed. Nonetheless, the inner portion preferably provides enough resistance to movement and a sufficiently strong engagement with the body passage to prevent the implantable occlusion device from moving with respect to the deployment site once deployed.

As noted above, the inner portion of an implantable occlusion device may be formed in a variety of manners (e.g., as known in the art), such as to form a scaffold or stent or frame structure. In some embodiments, the scaffold is formed from one or more members/elements (such terms being used interchangeably herein without intent to limit) combined to form a rigid and/or semi-rigid structure. The members may be formed of one or more struts, wires, strands, filaments, ribbons, etc., (such terms being used interchangeably herein without intent to limit) which are braided, interengaged, intertwined, interwoven, knitted, knotted, looped (e.g., bobbinet-style), weaved, woven, wrapped, or the like to form an expandable and contractable scaffold configuration. Alternatively, the members forming the implantable medical device may be formed by cutting (e.g., by laser-cutting) a structure (e.g., an, optionally monolithic, cylindrical tubular member) into an expandable configuration (e.g., a stent configuration with flanges on one or both ends), the cuts forming members such as strut members.

The members forming the inner portion of an implantable occlusion device formed in accordance with various principles of the present disclosure may be formed from a variety of non-limiting preferably biocompatible materials, such as, without limitation, a metal, metal alloy, polymer, metal-polymer composite, ceramics, and combinations or subcombinations thereof. For instance, the filaments may be formed from a variety of non-limiting preferably biocompatible metals, such as, without limitation, stainless steel, a nickel-titanium alloy such as Nitinol, a nickel-tungsten or tungsten alloy, a cobalt-chromium alloy, a cobalt-chromium-nickel based alloy such as Elgiloy®, a nickel-copper alloy, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and subcombinations and other alloys thereof. Additionally or alternatively, the members forming the implantable medical device may be formed from a variety of non-limiting preferably biocompatible polymers, such as, without limitation, polypropylene, polyester, polysulfone, nylon, silicones, polyurethane, polystyrene, polyethylene (PE) (including high-density and low-density PE's), polyethylene terephthalate (PET), polyethylene naphthalate (PEN), polybutylene terephthalate (PBT), polytrimethylene terephthalate, polyether block amides (PEBA), polyetheretherketone (PEEK), polyetherimide (PEI), poly(methyl methacrylate) (PMMA), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly paraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, subcombinations, and copolymers thereof. It will be appreciated that the members forming the implantable medical device may be formed from a mixtures, composites, combinations, subcombinations, copolymers, or co-constructions of any of the above. Provision of an outer portion over the inner portion may allow for a wider selection of materials for forming the inner portion, such as materials with lower levels of biocompatibility.

The inner portion of an implantable occlusion device formed in accordance with various principles of the present disclosure may be a self-expanding device such as known or heretofore known to those of ordinary skill in the art. For instance, the inner portion may be formed of shape-memory or heat-formable material (e.g., Nitinol or Elgiloy® or shape memory polymers) so that the inner portion returns to a pre-shaped expanded configuration from a collapsed delivery configuration upon advancement from a delivery sheath and/or withdrawal of a delivery sheath to deploy the implantable occlusion device. Such delivery sheath may be any acceptable tubular elongated member such as known to those of ordinary skill in the art for delivery of medical devices, and which may maintain the implantable occlusion device in a delivery configuration therein.

The outer portion may be configured to extend over the inner portion, such as to isolate the inner portion from the body passage (e.g., from direct contact with body passage). In some embodiments, the outer portion is a flexible and/or compliant/compliable cover portion. The outer portion may be formed from any of a variety of biocompatible materials. For instance, if implanted with respect to a dynamic anatomical site (e.g., a pylorus which constantly moves/shifts configuration), the outer portion may be formed of compliant or semi-compliant materials facilitating movement in response to movement of the anatomical site with respect to which the implantable occlusion device is deployed. In some embodiments, the outer portion has varying compliability along the length thereof, and in some instance may be more compliant along a portion thereof (e.g., more compliant at a location to be positioned in a pylorus than locations to be positioned on either side of the pylorus). The outer portion may isolate the body passage from any structural anomalies, degradation, wear, etc., of the inner portion. Examples of materials which may be used to form an outer portion of an implantable occlusion device formed in accordance with various principles of the present disclosure include polymeric materials such as, without limitation, silicones, polytetrafluoroethylene (PTFE), various block copolymers (e.g., block copolymers made of rigid polyamide blocks and soft polyether blocks, such as Pebax®), polyamides (e.g., Grilamid®, Vestamid®, etc.), polyurethane nylon elastomers, thermoplastic elastomers, etc. Such outer portion of an implantable occlusion device formed in accordance with various principles of the present disclosure may protect body tissue at the deployment site, and/or the integrity of the inner portion of the device. For instance, if a component of the inner portion degrades, the inner portion is not in direct contact with tissue. As such, any degradation or other unexpected or unintended changes to the inner portion (changes other than intended changes such as expansion or flexing) do not have an impact on tissue at the deployment site. As noted above, although biocompatible materials may be preferable for formation of the inner portion of an implantable occlusion device formed in accordance with various principles of the present disclosure, biocompatibility is a lesser concern if the outer portion isolates the inner portion from contact with body tissue.

The outer portion may envelop or enclose or encapsulate the inner portion. For instance, the other portion may be in the form of a balloon in which the inner portion is positioned. For instance, an implantable occlusion device may be deployed across a body passage (e.g., a pylorus) to be occluded. An outer portion in the form of a balloon may be inflated to occlude the body passage. The inner portion may include a retention member positionable within each end of the balloon and configured to retain the implantable occlusion device generally in place with respect to the body passage. The outer portion extends over the retention members between the retention members and the body tissue.

An implantable occlusion device formed in accordance with various principles of the present disclosure may include a shaft extending therethrough. The shaft may facilitate deployment of the implantable occlusion device. For instance, in embodiments in which the outer portion encloses the inner portion, the shaft may facilitate expansion of the outer portion for deployment across a body passage. In some embodiments, the shaft may be separable from an inflation lumen used to inflate an outer portion of the implantable occlusion device.

In some embodiments of an implantable occlusion device having a shaft therethrough, the inner portion may be axially movable with respect to the outer portion, such as by sliding along the shaft. Such movement or adjustability of the positions of the inner portion of the implantable occlusion device allows adjustability of the implantable occlusion device to accommodate deployment sites of varying sizes (lengths/widths/thicknesses). In some embodiments, the inner portion is in the form of a stent with modified retention members, such as retention members (e.g., flanges) of known stents but without the saddle member connecting the retention members. In some embodiments, the inner portion is the form of one or more, and preferably two or more, ring-shaped or toroidal-shaped or disk-shaped retention members. Such retention members may be spaced apart from each other within the outer portion of the implantable occlusion device, and may be separately formed from each other.

In some embodiments, an implantable occlusion device formed in accordance with various principles of the present disclosure has an outer portion formed of a compliant material, and an inner portion with retention members movable with respect to the outer portion. The outer portion may fully enclose the inner portion (e.g., may be in the form of a balloon). In an embodiment in which the outer portion is an enclosed structure such as a balloon, forces may be distributed substantially evenly on the outer surface of the outer portion, thereby reducing stress concentrations in small areas due to contraction of the implantable occlusion device, such as in response to anatomical forces exerted thereon. Upon placement across a dynamic body passage, such as a pylorus, the body passage may exert force on the implantable occlusion device which causes the retention members to move apart. The compliability of the outer portion allows the implantable occlusion device to conform to the body passage and to occlude flow of materials through the body passage. For instance, a compliable outer portion may be formed of a material which allows the compliable outer portion to “ride” waves of contractions (e.g., peristalsis) of the body walls at the deployment site, conforming with minimal resistance and thus not withstanding stress or strain which may otherwise weaken a less compliant material. As the outer portion may be compressed with contraction of the body wall at the deployment site, the inner portions may move apart. For instance, the inner portions may shift or extend to either end of the body passage, while anchoring the implantable occlusion device with respect to the body passage to resist migration of the implantable occlusion device with respect to the deployment site. In an embodiment with separately formed retention members (not coupled together with a saddle portion as in prior art stents), the outer portion holds the inner portions in place with respect to the deployment site.

Various embodiments of devices, systems, and methods for deployment at an anatomical site, such as for occluding a body passage, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

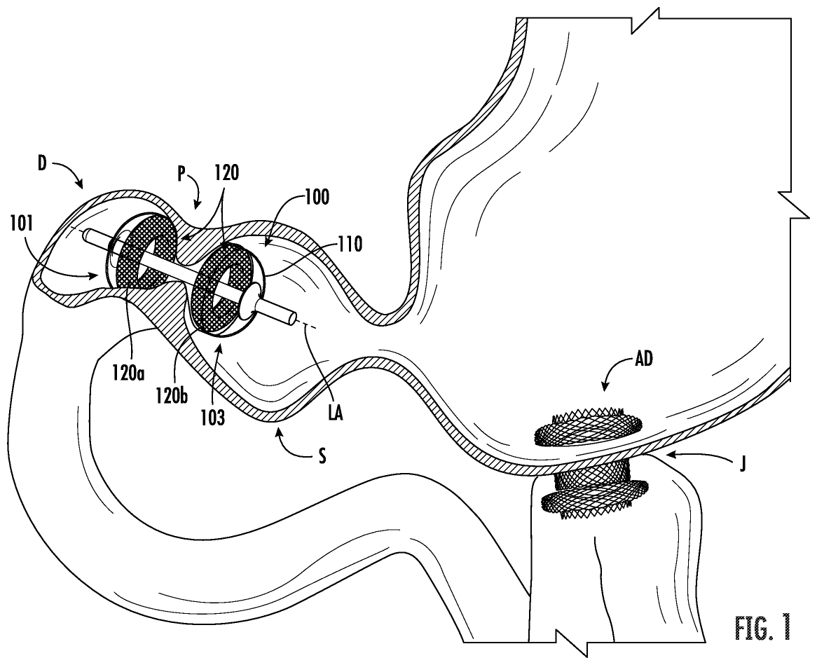

Turning now to the drawings, an example of an embodiment of an implantable occlusion device 100 formed in accordance with various principles of the present disclosure is illustrated in FIG. 1 in an example of a gastric environment. Specifically, the implantable occlusion device 100 is illustrated as deployed across a pylorus P, with a distal end 101 of the implantable occlusion device 100 positioned within a duodenum D and a proximal end 103 of the implantable occlusion device 100 positioned within a stomach S. In some embodiments, an additional implantable device, such as anastomosis device AD, is implanted in the vicinity of the implantable occlusion device 100. For instance, in the illustrated example of an embodiment, the anastomosis device AD creates an anastomosis between the stomach S and the jejunum J, and the implantable occlusion device 100 occludes flow of gastric materials into the duodenum D. With such arrangement, gastric materials are redirected from the stomach S to the jejunum J (rather than to the duodenum D) through the anastomosis device AD.

In accordance with various principles of the present disclosure, the example of an embodiment of an implantable occlusion device 100 illustrated in FIG. 1 has an outer portion 110 and an inner portion 120 positioned within the outer portion 110. More particularly, in the illustrated example of an embodiment, the inner portion 120 includes two or more portions 120a, 120b which may be movable with respect to each other and with respect to the outer portion 110. For example, the inner portions 120a, 120b may shift axially along the longitudinal axis LA of the implantable occlusion device 100 during natural movements of the implantable occlusion device 100, such as during peristalsis (as deployed, for example, across a pylorus P). In the illustrated example of an embodiment, the outer portion 110 extends across the deployment site with one portion 120a of the inner portion 120 on a distal side of the pylorus P, and one portion 120b of the inner portion 120 on a proximal side of the pylorus P. The pylorus P may close onto the implantable occlusion device 100, such as by exerting a radially-inwardly directed force on the implantable occlusion device 100. The outer portion 110 may be compressed radially-inwardly along a portion thereof between the inner portions 120a, 120b, and the inner portions 120a, 120b may be driven apart in response to the radially-inwardly directed force exerted by the pylorus P. In accordance with various principles of the present disclosure, the inner portions 120a, 120b are configured to anchor the implantable occlusion device 100 with respect to the deployment site to resist migration of the implantable occlusion device 100 with respect to the deployment site.

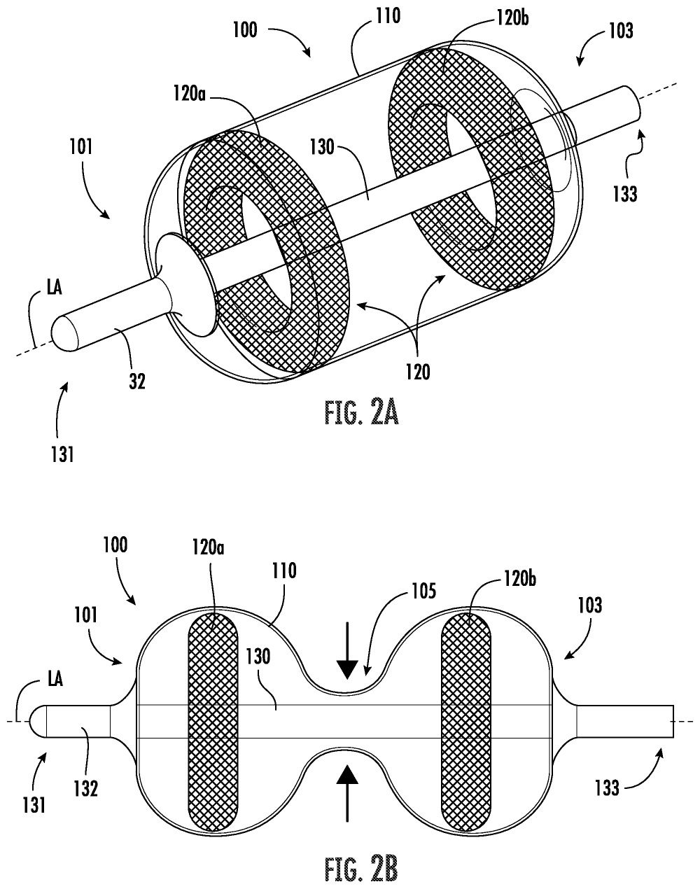

An example of an embodiment of an implantable occlusion device 100 such as illustrated in FIG. 1 is illustrated in FIG. 2A and FIG. 2B. The implantable occlusion device 100 is shown in a generally neutral configuration in FIG. 2A. In FIG. 2B, the implantable occlusion device 100 is shown with a radially-inwardly directed force (indicated schematically by the illustrated arrows) applied to a medial section 105 of the implantable occlusion device 100 (between the distal end 101 and the proximal end 103 of the implantable occlusion device 100). As may be appreciated with reference to FIG. 2A and FIG. 2B, the outer portion 110 is sufficiently flexible or compliant to flex inwardly upon the application of force to a medial section 105 of the illustrated implantable occlusion device 100. Moreover, the inner portion 120 may be movable with respect to the outer portion 110 to be moved towards either end 101, 103 of the implantable occlusion device 100 upon application of a force to the medial section 105 of the implantable occlusion device 100. As may be appreciated with reference to FIG. 1 and FIG. 2B, the inner portions 120a, 120b may be advantageously positioned at the ends of the deployment site (e.g., in the stomach S upstream of the pylorus P, and in the duodenum D downstream of the pylorus P) and seated against anatomical structures to resist migration of the implantable occlusion device 100 with respect to the deployment site (such as in a manner known to those of ordinary skill in the art).

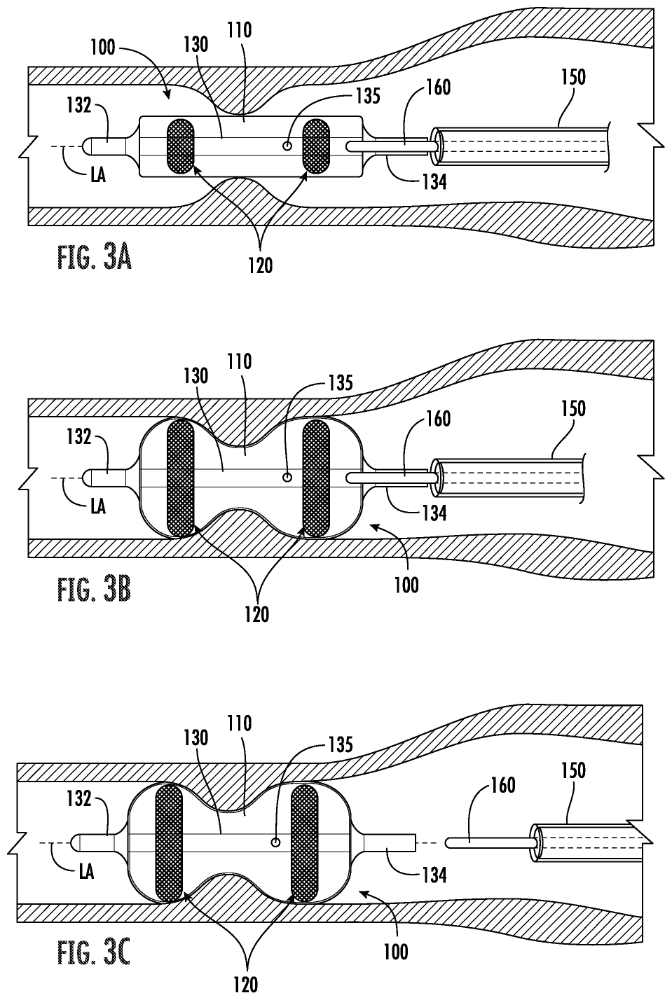

In the example of an embodiment illustrated in FIG. 2A and FIG. 2B, the outer portion 110 is formed as a balloon, and the inner portion 120 is formed as two separate inner portions 120a, 120b movable with respect to each other as well as with respect to the outer portion 110 (e.g., independently slidable along the longitudinal axis LA of the implantable occlusion device 100). The inner portions 120a, 120b may be formed as ring-shaped or toroidal-shaped retention members which are expandable from a delivery configuration to the illustrated deployed configuration. A delivery configuration of the implantable occlusion device 100 (including the outer portion 110 and the inner portion 120) is shown schematically in FIG. 3A, with the implantable occlusion device 100 in a contracted configuration as initially deployed at the deployment site. The implantable occlusion device 100 may be further contracted for delivery within the delivery shaft 150 to the deployment site. The delivery shaft 150 may be capable of transluminal navigation through a body, such as through the gastrointestinal system. In the deployed configuration, such as illustrated in FIG. 2A and FIG. 2B, the inner portion 120 may expand the outer portion 110 (such as if the inner portion 120 is self-expanding upon deployment from the delivery shaft 150) or may simply expand with expansion of the outer portion 110 (e.g., inflation of an outer portion 110 in the form of an inflatable balloon, as discussed in further detail below with respect to FIG. 3B and FIG. 3C). The outer surface of the inner portion 120 (e.g., defined by the outer diameter of the inner portions 120a, 120b) may contact the interior of the inner portion 120 and, via the outer portion 110, engage or seat against the body wall at the deployment site. In some embodiments, the inner portion 120 supports the outer portion 110.

The example of an embodiment of an implantable occlusion device 100 illustrated in FIG. 2A and FIG. 2B may include a shaft or mandrel 130 extending generally axially therethrough along a longitudinal axis LA of the implantable occlusion device 100. In some embodiments, the outer portion 110 is formed as a balloon formed over and coupled to the distal end 131 and the proximal end 133 of the mandrel 130. As such, the ends of the outer portion 110 are sealed to the mandrel 130 and the outer portion 110 may be inflatable to expand the implantable occlusion device 100.

The distal end 131 of the mandrel 130 may form a guide tip 132 (optionally with the outer portion 110 formed thereover) to facilitate guiding of the implantable occlusion device 100 to a deployment site without the need of a guide wire. As may be appreciated, if the implantable occlusion device 100 is to be deployed and left in place within a body passage, and the outer portion 110 is to be inflated, a typical configuration of a balloon catheter, with a catheter having an inflation lumen as well as a guidewire lumen coupled to the balloon, may not be feasible. Formation of the distal end 131 of the mandrel 130 as a guide tip 132 allows insertion and delivery of the implantable occlusion device 100 without the assistance of a separate guide wire.

The proximal end 133 of the mandrel 130 may form an inflation port 134 for fluidly coupling with an inflation lumen 160, such as illustrated in FIG. 3A and FIG. 3B. Once the implantable occlusion device 100 is deployed out of a delivery shaft 150 (as illustrated, for example, in FIG. 3A), the outer portion 110 may be inflated via the inflation lumen 160, such as illustrated in FIG. 3B. Once the implantable occlusion device 100 has been sufficiently expanded to be deployed (schematically illustrated in FIG. 3B), the inflation lumen 160 may be decoupled from the inflation port 134, such as illustrated in FIG. 3C, to leave the implantable occlusion device 100 in place at the deployment site. It will be appreciated that the delivery shaft 150, inflation lumen 160, and inflation port 134 may be formed in any desired manner known to those of ordinary skill in the art to achieve inflation of an expandable implantable deployable device such as an implantable balloon. For instance, the mandrel 130 may include one or more inflation apertures 135 in fluid communication with the inflation port 134 and the inflation lumen 160. The inflation port 134 may be self-sealing so that once the inflation lumen 160 is separated therefrom, the inflation port 134 closes to hold inflation medium (e.g., saline) within the inflated outer portion 110 of the implantable occlusion device 100.

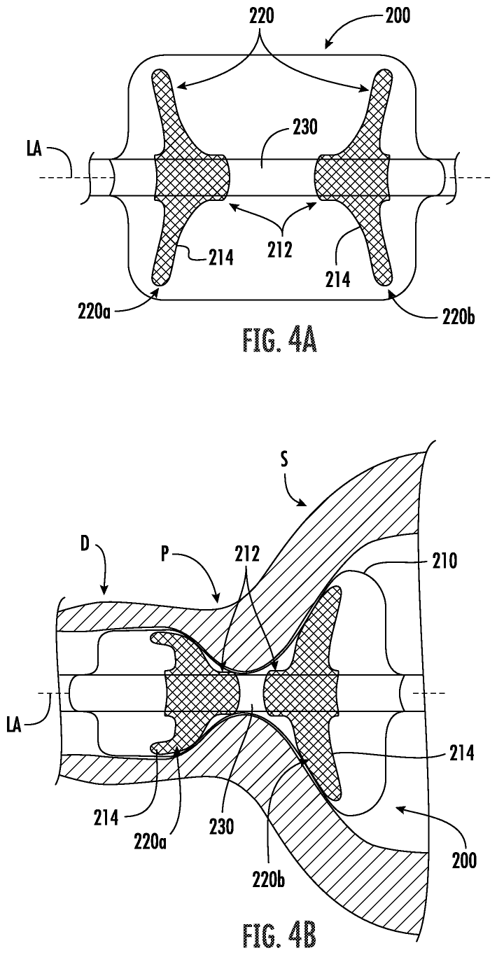

It will be appreciated that other configurations of implantable occlusion devices with an outer portion and an inner portion are within the scope and spirit of the present disclosure. For instance, instead of having an inner portion being in the general shape of tori or rings, an implantable occlusion device 200 may have an inner portion 220 with one or more disk-shaped retention members 220a, 220b. Such configuration of inner portions 220a, 220b allows a greater radial extent of the inner portions 220a, 220b from a generally axially-centrally positioned hub 212 along radially-extending flanges 214 to the interior of the outer portion 210. Such increased radial extent of the inner portion 220 allows the inner portion 220 to conform to the anatomy on either side of the body passage across which the outer portion 210 extends (and to which the outer portion 210 conforms). For instance, the outer portion 210 may conform a portion of the shape of the implantable occlusion device 200 with respect to a body passage, and the inner portion 220 may conform end portions of the implantable occlusion device 200 with respect to anatomical walls at either end of the body passage to anchor the implantable occlusion device 200 with respect to the deployment site to resist migration of the implantable occlusion device 200 with respect to the deployment site. For instance, as illustrated in FIG. 4B, a flange 214 of one retention member 220a may be positioned in a duodenum D and may generally conform to the duodenal side of a pylorus P, while a flange of another retention member 220b may be positioned in a stomach S and may generally conform to the gastric side of the pylorus P. It will be appreciated that the flanges 214 need not be symmetrical (as illustrated). For instance, one flange 214 may be configured to conform to the anatomical structure at one end of the deployment site (e.g., the duodenal anatomy) and the other flange 214 may be configured to conform to the anatomical structure at the other end of the deployment site (e.g., the gastric anatomy). As in the example of an embodiment illustrated in FIG. 2A and FIG. 2B, the inner portions 220a, 220b may be movable with respect to each other, such as independently slidable along the longitudinal axis LA of the implantable occlusion device 200.

It will be appreciated that in an embodiment in which a mandrel 130, 230 extends through the implantable occlusion device 100, 200, the inner portion 120, 220 may have a radially-inwardly positioned section either spaced apart from the mandrel 130 (as in the case of the inner portion 120 of the implantable occlusion device 100), or generally positioned closer with respect to the mandrel 230 (as in the case of the inner portion 220 of the implantable occlusion device 200). For instance, an inner portion 120 in the form of a torus, as in FIG. 2A and FIG. 2B, may have an inner diameter larger than and spaced apart from an outer diameter of a mandrel 130 about which the inner portion 120 is mounted. In contrast, an inner portion 220 in the form of retention members 220a, 220b as in FIG. 4A and FIG. 4B may have a collar portion or hub 212 more closely positioned with respect to the mandrel 230 (or at least in closer proximity than the inner diameter of the inner portion 120 in FIG. 2A and FIG. 2B are positioned with respect to the mandrel 130). It will be appreciated that the hubs 212 may nonetheless be positioned with respect to the mandrel 230 to slide with respect thereto. The flanges 214 of an inner portion 220 as illustrated in FIG. 3A and FIG. 3B may provide additional structure area over which the inner portion 220 may anchor the implantable occlusion device 200 with respect to the deployment site.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. It will be appreciated that various aspects of the above disclosure may be applied in other passages within the body to reduce flow through such passage or otherwise.

Various further benefits of the various aspects, features, components, and structures of an implantable device and/or system, and various aspects of use and/or manufacture thereof, such as described above, and in addition to those discussed above, may be appreciated by those of ordinary skill in the art.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable device configured to be delivered to and deployed at a deployment site, said implantable device comprising:
an inner scaffold portion shiftable between a delivery configuration and an expanded deployed configuration; and
an outer portion formed separately from said inner portion and positioned over said inner portion such that, upon deployment, said outer portion is positioned between a body wall at the deployment site and said inner portion of said implantable device.

2. The implantable device of claim 1, wherein said inner portion comprises a first inner portion and a second inner portion.

3. The implantable of claim 2, wherein said first inner portion and said second inner portion are movable with respect to said outer portion and with respect to each other.

4. The implantable of claim 3, wherein said first inner portion and said second inner portion are movable away from each other in response to force applied to said outer portion.

5. The implantable device of claim 1, wherein said outer portion is a balloon enclosing said inner portion.

6. The implantable device of claim 5, wherein an end of said balloon is tapered to guide said implantable device to a deployment site.

7. The implantable device of claim 5, wherein said balloon includes an inflation valve separably couplable with an inflation lumen to allow inflation of said balloon and separation from the inflation lumen upon deployment of said balloon.

8. The implantable device of claim 1, wherein said inner portion comprises one or more ring-shaped retention members.

9. The implantable device of claim 8, wherein said inner portion comprises first and second toroidal retention members.

10. The implantable device of claim 1, wherein said inner portion comprises one or more disk-shaped retention members.

11. The implantable device of claim 1, further comprising a shaft extending through said implantable device.

12. The implantable device of claim 11, wherein said shaft extends axially within said implantable device, and said inner portion comprises a first inner portion and a second inner portion movable with respect to said shaft in response to radially-inwardly directed force applied to said implantable device when said implantable device is deployed at a deployment site.

13. The implantable device of claim 11, wherein said inner portion comprises one or more disk-shaped retention members having collars mounted on said shaft and movable with respect to said shaft.

14. The implantable device of claim 1, wherein said outer portion is expandable upon shifting of said inner portion from the delivery configuration to the deployed configuration.

15. A method of forming an implantable occlusion device, said method comprising:
mounting one or more retention members over a shaft;
positioning a compliable cover portion over the retention members and the shaft; and
sealing the cover portion with respect to the shaft at a proximal end and a distal end to enclose the retention members therein.

16. The method of claim 15, further comprising mounting the one or more retention members slidably over the shaft.

17. A method of occluding a body passage, said method comprising:
delivering an implantable occlusion device to the body passage, the implantable occlusion device having an inner scaffold portion and an outer portion enclosing the inner portion;
expanding the outer portion to engage the wall of the body passage to deploy the implantable occlusion device and to occlude the body passage; and
allowing the inner portion to shift to an expanded deployed configuration on either side of the body passage to anchor the implantable occlusion device with respect to the body passage to resist migration from the deployment site.

18. The method of claim 17, further comprising deploying the implantable occlusion device across a pylorus.

19. The method of claim 17, wherein the outer portion is a compliant balloon and the inner portion includes first and second inner portions, said method comprising deploying the compliant balloon at a deployment site which applies radially-inwardly directed force to the compliant balloon to compress a section of the compliant balloon and to cause the first and second inner portions to move apart from each other axially.

20. The method of claim 17, wherein the outer portion is a balloon, said method further comprising inflating the balloon with an inflation lumen when the balloon is delivered to the deployment site, and removing the inflation lumen from the balloon to deploy the implantable occlusion device at the deployment site.
